(19) United States
(12) Patent Application Publication
(10) Pub. No.: US 2024/0277514 A1
(43) Pub. Date: Aug. 22, 2024

(54) Heated, Torso Wrap Device, System and Method

(71) Applicant: Forza Innovations Inc., San Diego, CA (US)

(72) Inventors: Johnny Forzani, Calgary (CA); Wei Cao, Arcadia, CA (US)

(21) Appl. No.: 18/172,923

(22) Filed: Feb. 22, 2023

(51) Int. Cl. A61F 7/00; A61H 7/00

(52) U.S. Cl. CPC A61F 7/007; A61H 7/001; A61F 2007/0018; A61F 2007/003; A61F 2007/0228; A61H 2201/0207; A61H 2201/1652; A61H 2205/08

(57) ABSTRACT

The present invention discloses a unitary, multizone, torso wrap, heat treatment system. Embodiments disclose two or more wraps for simultaneously or selectively heating two or more zones of a torso. The invention also discloses novel structures and methods for adjusting the specific locations of one or more heat wraps on a person's torso.

### BACKGROUND OF THE INVENTION

The present invention pertains to body wraps and more particularly to wraps that supply heat therapy to ailing torso areas of a human body.

Many people suffer from temporary or chronic back and shoulder pain. This is due to any number of reasons, including trauma from injury, stresses, habit, improper body mechanics, exercise and age. Of course, merely saying that a person suffers from back or shoulder pain does not identify where on or along the back or shoulder the person is suffering, and whether it is structurally related to some part of the spinal cord, the muscular system, tissue, or tendons.

For example, more than thirty bones, i.e., vertebrae, combine to form the spinal column, also called the spine, which houses the spinal cord. Spacing between the vertebrae are maintained by intervertebral cartilage discs which allow for spine flexure and general cushioning of the spinal structure and its components. Indeed, pain can emanate from any spot along the spine.

More specifically, the spine comprises four regions. First are the seven cervical or neck vertebrae, generally designated C1-C7; second, the 12 thoracic or upper back vertebrae, generally designated T1-T12; third, the five lumbar or lower back vertebrae, generally designated L1-L5; and fourth, the sacrum and coccyx, s called the “tail bone,” which are fused together and form the base of the spine.

It is thus understood that a person may suffer from spinal back pain in any one or more of these regions. Neck pain (at the cervical vertebrae), often expressed as shoulder pain, upper back pain, Low back pain, and tail bone pain are all common ailments that need treatment. Indeed, the vast majority of back pain patients are diagnosed with nonspecific acute back pain which has no serious underlying pathology and often patients cannot pinpoint a single identifiable region of pain. That said, low back pain is one of the more common regions people suffer with.

Shoulder pain is another common medical problem caused by numerous conditions. For example, subacromial impingement syndrome (SIS) is one of the most common causes of shoulder pain. Anatomically, SIS refers to the supraspinatus tendon impinging on the undersurface of the anterior acromion as the arm is raised overhead. Typically, pain is generated with elevation of the arm above the head though it can occur with rest. Multiple pathologies, such as subacromial bursitis, rotator cuff tendinopathy, partial rotator cuff tears, and even small tears can coexist to create SIS.

The above back, neck and shoulder regions of the body can also experience temporary soreness from any of myriad reasons, including strenuous resistance training. For these ailments, treatment is also desirable. A wide variety of well-known, non-invasive conventional interventions are available for symptomatic relief and/or treatment of these pains. These include, without limitation, application of compresses, bed rest, massages, self-stretching exercise, medications, and spinal manipulation with a physical therapist or chiropractor. Less conventional approaches to symptomatic relief and treatment include, but are not limited to acupuncture, biofeedback, interventional therapy, traction, transcutaneous electrical nerve stimulation (TENS), and ultrasound.

One popular, low risk category of solutions that are effective to relieve and even prevent back and shoulder pain is the numerous back and torso heat-supplying and compression support devices, commonly in the form of a wrap or “belt”. Other devices such as generic, at-home heating pads are also often used by simply resting them on regions of the torso.

Unfortunately, existing devices that supply heat or heat and compression are designed to treating one specific part of a body, or one part at a time. Thus, if a person is suffering pain simultaneously in more than one region, say in the lower back and shoulder, her options are limited by the devices that treat only one region at a time.

Accordingly, the art of non-invasive heat treating back and shoulder, hereinafter “torso,” pain therapy would benefit from a device and system that simultaneously treats multiple ailing regions of a person's body. The present invention addresses this need and more.

### SUMMARY OF THE INVENTION

The present invention employs a system for heat treating zones of a person's body. In preferred embodiments, a multizone, preferably unitary, wrap system for supplying heat treatment to a torso of a wearer is disclosed. The system includes a lower torso wrap adapted to hold one or more heating elements for placement against at least a part of a wearer's lower torso region and for supplying heat to that part of the wearer's lower torso region when worn; and a second wrap spaced apart from and above the lower torso wrap and connected to the lower torso wrap, the second wrap adapted to hold one or more heating elements and for placement against and supplying heat to a second region of the wearer's torso when worn.

In further embodiments, the system includes a spine connecting the lower torso wrap and the second wrap. The lower torso and second wraps may be secured to wearer's torso by wrapping them around the wearer's torso. In preferred embodiments, the second wrap may be movable up and down the spine to adjustably set the location of the second wrap corresponding to a desired spot on the torso of the wearer. In another embodiment, the second region of the wearer's torso is a shoulder region, and the second wrap is a heat-supplying shoulder strap.

In yet another configuration of the present invention the system further includes a third wrap spaced apart from and above the second torso wrap and adapted to hold one or more heating elements. The third wrap is for placement against and supplying heat to a third region of the wearer's torso when worn. In this configuration, the third wrap may also be movable up and down the spine to adjustably set its location corresponding to a desired spot on the torso and above the second wrap. Optionally, the second wrap may also be movable up and down the spine to adjustably set the location of the second wrap corresponding to a desired spot on the torso. This embodiment may also include a shoulder panel adapted to hold one or more heating elements, where the shoulder panel is for placement against and supplying heat to a shoulder region of the wearer's torso when the wrap system is worn.

In further optional details of the present invention, the one or more heating elements in the lower torso and second wraps may be integral with the wraps. Alternatively, the heating elements may be removable from the wraps and are adapted to be heated external to the system. The heating elements may be powered by one or more batteries connected to the wrap system. Preferably, the first and second wraps are adapted to supply compression forces on the regions of the torso the wraps engage.

In other embodiments of the invention, an adjustable heat wrap for a torso is disclosed. The wrap includes a heating wrap for wrapping around the torso of the wearer, and a pair of adjustable length shoulder straps, each strap having ends connected to the upper edge of opposite ends of the wrap, such that by adjusting the length of the shoulder straps, the heating wrap may be adjustably wrapped around a desired region of the wearer's torso.

A method for heat treating zones of a subject's torso is also disclosed by the present invention. In embodiments, the method includes the steps of first donning onto the torso an integrated, multizone, wrap system having a lower torso wrap and a second torso wrap spaced apart from and above the lower torso wrap, both wraps having therein one or more heating elements; then adjusting the location of the second torso wrap to a desired zone on the torso; securing the lower torso wrap and second torso wrap onto the torso; and electronically activating the wrap system to supply heat to the torso. In preferred embodiments, the activation of the wrap system supplies heat simultaneously from the lower torso wrap and second torso wrap to the torso, and the activation of the wrap system supplies heat selectively from the lower torso wrap and second torso wrap to the torso.

### BRIEF DESCRIPTION OF THE FIGURES

Further advantages of the present invention may become apparent to those skilled in the art with the benefit of the following detailed description of the preferred embodiments and upon reference to the accompanying drawings in which:

FIG. 1 is a front perspective view of one embodiment of the heat wrap system present invention shown as worn by a person;

FIG. 2 is a rear perspective view of the system shown in FIG. 1 as worn by a person;

FIG. 3 is perspective view of a portion of the system shown in FIGS. 1 and 2 illustrating the lower torso panel;

FIG. 4 is perspective view of the heating element of lower torso panel shown in FIG. 3, according to one embodiment;

FIG. 5 is a front perspective view of a second embodiment of the heat wrap system present invention shown on an illustrative torso;

FIG. 6 is a rear perspective view of the system shown in FIG. 5;

FIG. 7 is perspective view of a third embodiment of the heat wrap system present invention;

FIG. 8. is a perspective view of an exemplary closure mechanism for securing a heat panel to the spine of a torso wrap system according to one embodiment of the present invention;

FIG. 9 is a perspective view of a rechargeable heating element that may be installed in a panel of a system of the present invention in accordance with one embodiment; and

FIG. 10 is a perspective view of a fourth embodiment of the heat wrap system present invention.

### DETAILED DESCRIPTION OF THE INVENTION

Referring now to the drawings, like reference numerals designate identical or corresponding features throughout the several views.

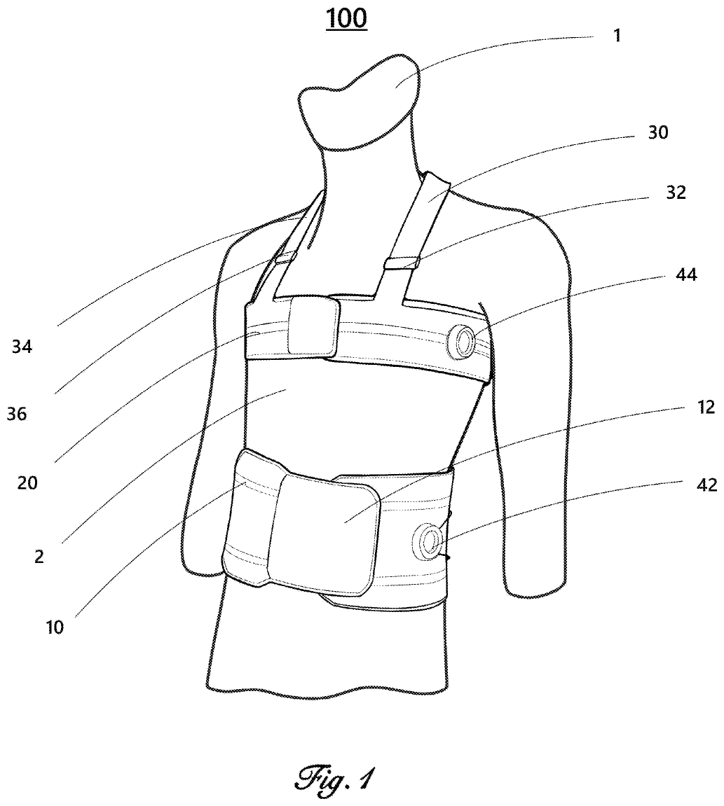

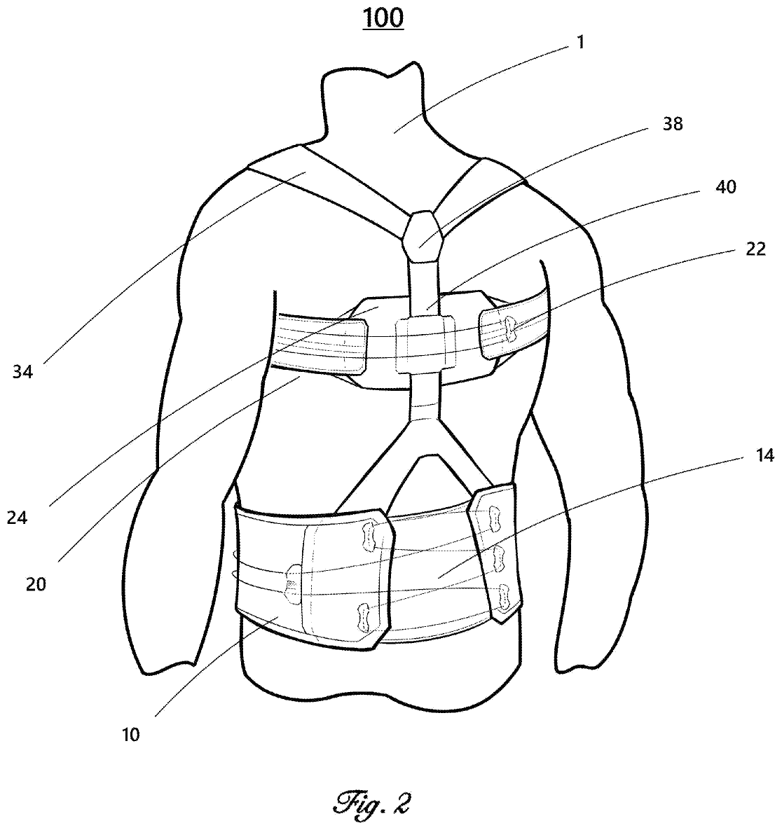

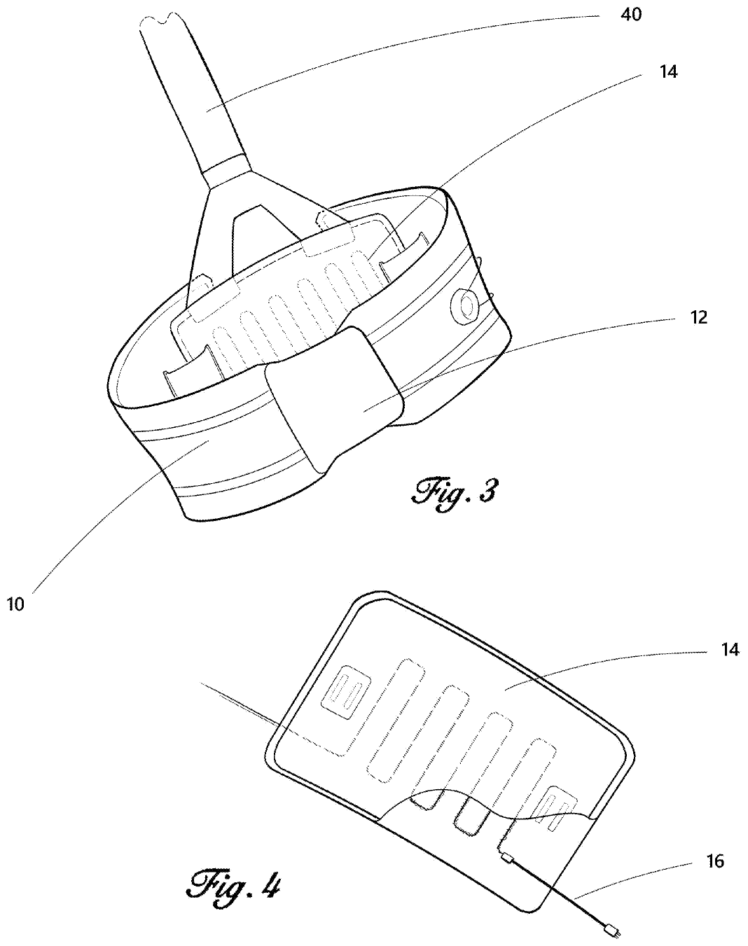

The drawings show a number of features of the present inventive heat wrap device and system. FIGS. 1 and 2 show perspective views of one multizone, here, dual location, heat wrap system with FIG. 3 showing a close-up view of the lower torso panel 10 and FIG. 4 showing a close-up view of the heating element 14 that is, in this embodiment, permanently installed (e.g., sewed or sealed) installed with a battery in a panel and rechargeable via plug 16. The remaining figures show alternative embodiments of the present invention.

Turning to FIGS. 1 and 2, shown is one preferred embodiment of a heat wrap system 100 having two wraps 10, 20 for supplying pain relieving heat therapy to two regions or zones of torso 2 of person 1. In particular, wrap 10 wraps around and secures to a lower torso region of person 1 to firmly place heating element 14 at a comfortable spot over the waist and lower back region. Wrap 10 may be firmly secured to this lower part of the torso 2 by securely closing the opposite ends of wrap 10 together at front pocket 12 using any appropriate closing mechanism such as hook and loop (e.g, Velcro™).

Wrap 20 is spaced apart from and positioned over wrap 10 to treat another part of the wearer's torso. As seen in FIGS. 1 and 2, wrap 20 is positioned at the chest in front and at the shoulder blade area at the back of person 1, just below person 1's underarms. In this embodiment, upper wrap 20 is also supported with shoulder straps 30, 34, each having adjustable lengths via buckles 32, 36 respectively. Straps 30, 34 meet at the back side of wearer 1 at junction 38. With this preferred structure, heating element 24 is securely positioned over person 1's spine in the middle of the back to treat that part of torso 2.

As further seen in FIG. 1, once wraps 10 and 20 are closed onto torso 2, the user 1 may adjustably tighten and loosen wraps 10, 20 using Boa turning dials 42, 44 installed on these wraps, respectively. It will be understood by those skilled in the art how to implement such dial and wire systems. In the present embodiment, wraps 10 and 20 are also connected to each other at the back via spine 40, which itself is connected to straps 30, 34 at junction 38 (FIG. 2). Spine 40 may take any suitable form. For example, it may be rigid, or it may be soft and pliable or bendable. Spine 40 may also be located at somewhere other than in the middle of the back when worn.

It should also be understood that spine 40 is only one means for connecting the two wraps according to the present invention. Other means and mechanisms for connecting the two wraps so that the system or device is a single integrated unit can be implemented. For example, in place of spine 40, the upper wrap 20 may be permanently (or not permanently) connected to wrap 10 with a soft, pliable material, such as a breathable cloth or mesh fabric. This embodiment can enable the system to collapse into a relatively small package, which may be preferable when space saving is desired, such as for shipping or for travel. With any of these configurations, it is readily apparent that person 1 can be effectively “heat treated” at two torso spots or locations simultaneously.

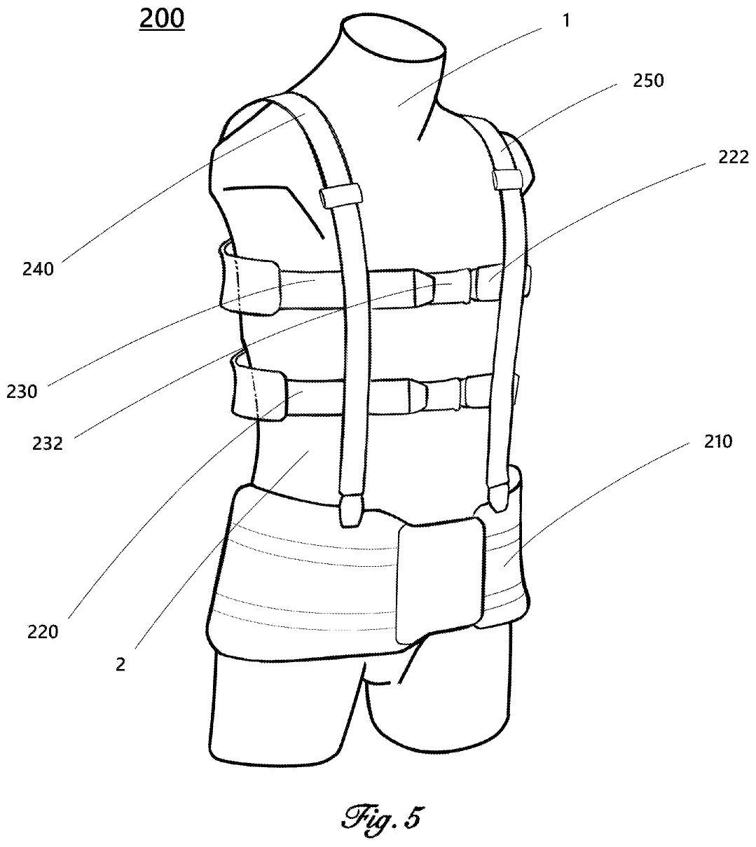

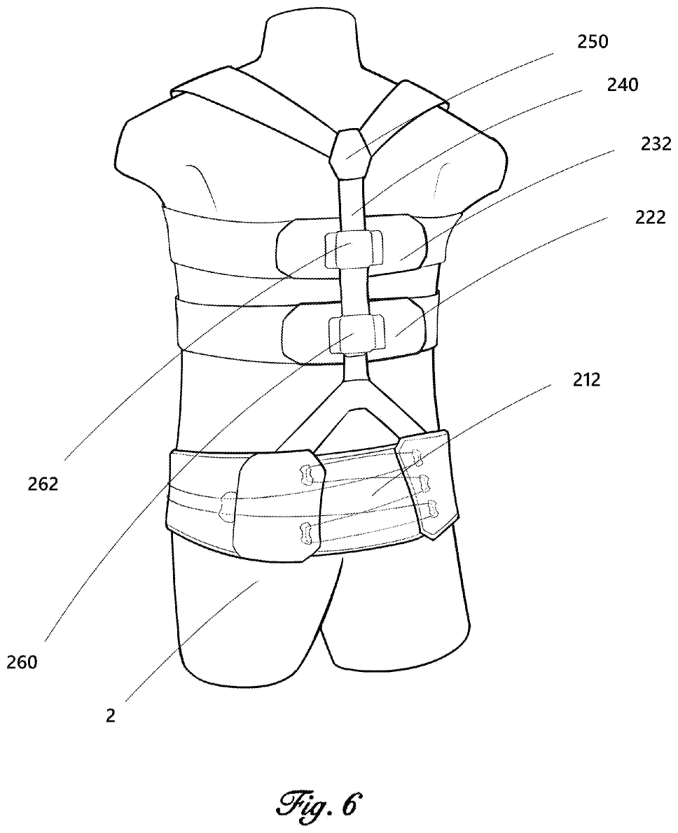

Turning now to another embodiment of the present invention, FIGS. 5 and 6 show a torso heat treatment system 200 that treats three regions of person 1′ simultaneously. Accordingly, system 200 includes a lower torso panel wrap 210, middle torso panel wrap 220 and upper torso panel wrap 230. As an alternative to the previous embodiment, shoulder straps 240, 250 are connected at the front side of, and secure the position of, lower torso panel wrap 210. At the rear side, these straps merge together and with spine 240 at junction 250. Now, as seen in FIG. 6, heating elements 212, 222 and 232 of wraps 210, 220 and 230 respectively, can simultaneously supply heat treatment to three (3) regions of the back.

Because in this embodiment, middle torso panel wrap 220 and upper torso panel wrap 230 are not supported by shoulder straps, it is important that these wraps are firmly secured to their intended locations on torso 2′. Thus, in a presently preferred example, and by way of example and not limitation, the wrap 220 is securely closed in front at buckle 222 and in the back, on spine 240 with closure 260. A close-up of this closure mechanism is shown in FIG. 8. Similarly, upper wrap 230 is securely closed in front at front buckle 232 and in the back, on spine 240 with closure 262.

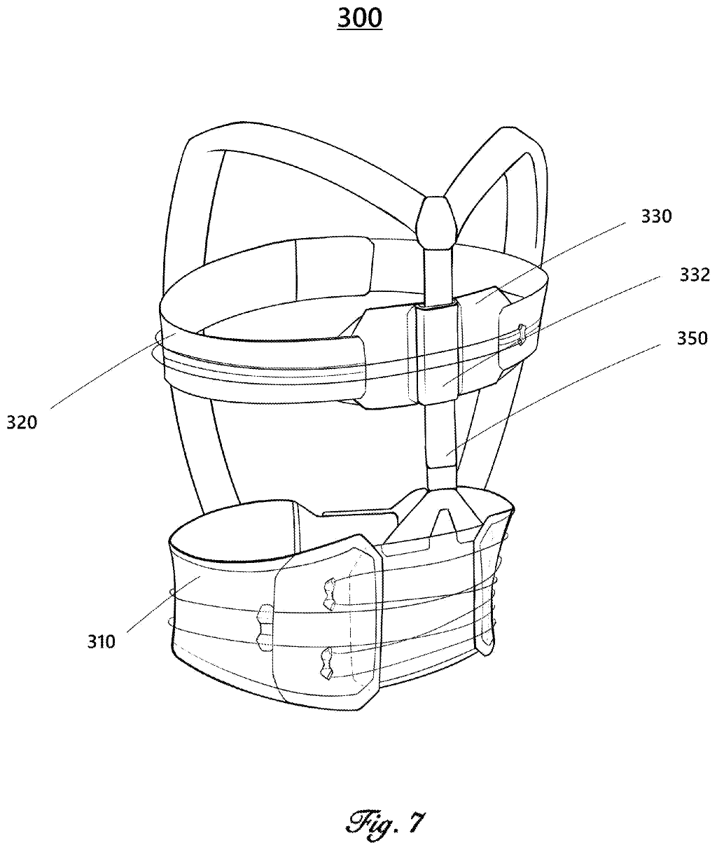

Turning to yet another embodiment of the present invention, FIG. 7 shows an alternative dual-location torso treatment wrap system 300. Here, lower torso wrap 310 is similar to lower torso wrap 10 in FIG. 1. However, upper torso wrap 320 has heating element 330 with channel 332 through which spine 350 resides. This robust spine/channel system enables upper torso wrap 320 to be precisely an securely adjusted up and down spine 350 until the wearer decides exactly where she wants the upper torso wrap heat treatment simultaneously with lower torso heat treatment from lower torso wrap 310.

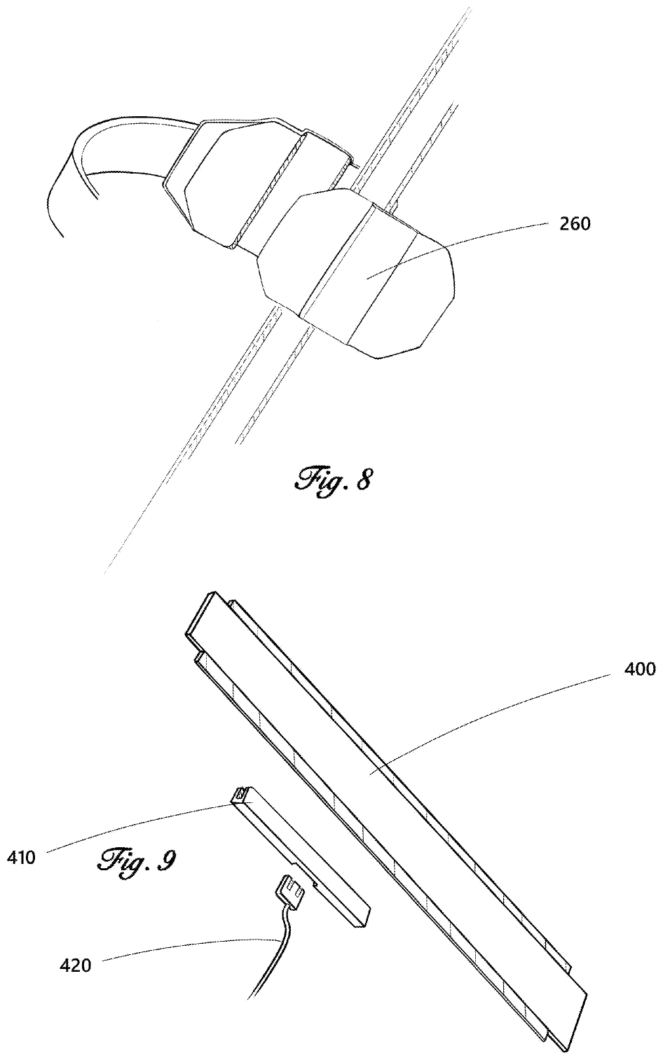

FIG. 9 shows an exploded view of one embodiment of the heating element system of the present invention that may be installed into the wraps. Shown here, heating element 400 in the form of a strip, has electronic unit 410 which may have a rechargeable battery built therein, with a plug unit 420 enable power to be connected thereto to recharge the battery in unit 410.

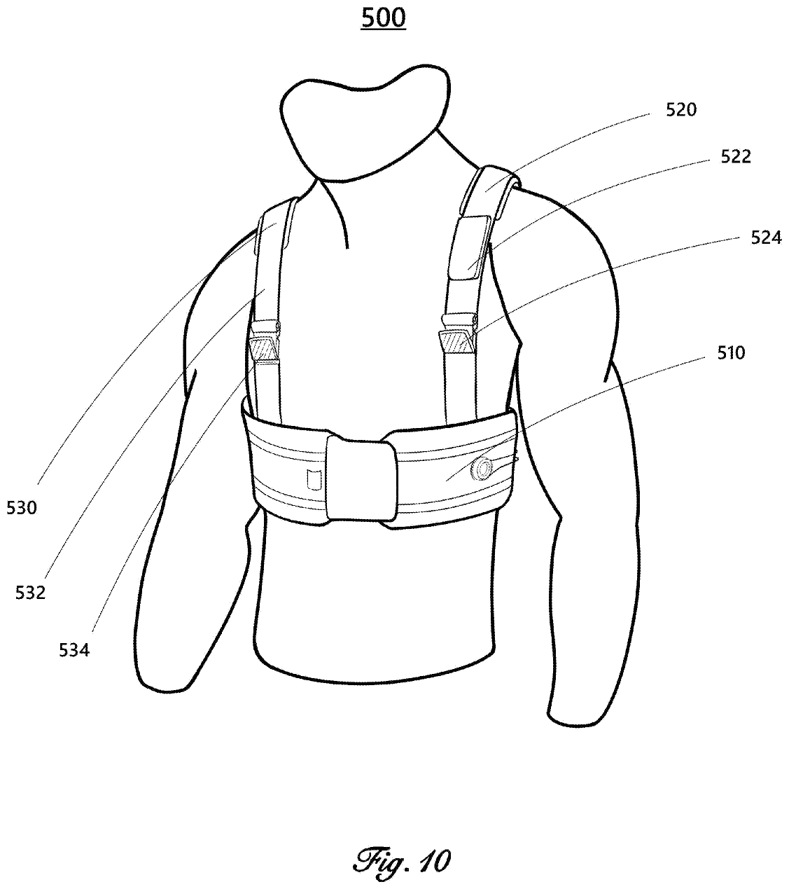

FIG. 10 shows a further alternative adjustable wrap system 500 structure as worn by person 1″ that is within the scope of the present invention. This embodiment shows a single wrap and shoulder strap structure that is highly adjustable. In particular, heat wrap 510 is connected to straps 520, 530 both at the front of the unit and in the back (not shown). Straps 520, 530 have heating elements 522, 532 respectively, that supply heat treatment to the shoulder areas. Straps 520, 530 also have length adjusting buckles 524, 534 respectively, for easy adjustment of the length of the straps while wearing the unit.

While embodiments of the invention have been illustrated and described, it is not intended that these embodiments illustrate and describe all possible forms of the invention. Various changes, modifications, and alterations in the teachings of the present invention may be contemplated by those skilled in the art without departing from the intended spirit and scope thereof. It is intended that the present invention encompass such changes and modifications.

1. A multizone, wrap system for supplying heat treatment to a torso of a wearer, comprising:
a. a lower torso wrap adapted to hold one or more heating elements for placement against at least a part of a wearer's lower torso region and for supplying heat to that part of the wearer's lower torso region when worn; and
b. a second wrap spaced apart from and above the lower torso wrap and connected to the lower torso wrap, the second wrap adapted to hold one or more heating elements and for placement against and supplying heat to a second region of the wearer's torso when worn.

2. The system of claim 1, further including a spine connecting the lower torso wrap and the second wrap.

3. The system of claim 1, wherein the lower torso and second wraps are secured to wearer's torso by wrapping them around the wearer's torso.

4. The wrap system of claim 2, wherein the second wrap is movable up and down the spine to adjustably set the location of the second wrap corresponding to a desired spot on the torso of the wearer.

5. The wrap system of claim 1, wherein the second region of the wearer's torso is a shoulder region, and the second wrap is a heat-supplying shoulder strap.

6. The wrap system of claim 1, further including a third wrap spaced apart from and above the second torso wrap and adapted to hold one or more heating elements, the third wrap for placement against and supplying heat to a third region of the wearer's torso when worn.

7. The wrap system of claim 6, wherein the third wrap is movable up and down the spine to adjustably set its location corresponding to a desired spot on the torso and above the second wrap.

8. The wrap system of claim 7, wherein the second wrap is movable up and down the spine to adjustably set the location of the second wrap corresponding to a desired spot on the torso.

9. The wrap system of claim 6, further including a shoulder panel adapted to hold one or more heating elements, the shoulder panel for placement against and supplying heat to a shoulder region of the wearer's torso when the wrap system is worn.

10. The wrap system of claim 1, wherein the one or more heating elements in the lower torso and second wraps are integral with the wraps.

11. The wrap system of claim 1, wherein the one or more heating elements are removable from the wraps and are adapted to be heated external to the system.

12. The wrap system of claim 1, wherein the heating elements are powered by one or more batteries connected to the wrap system.

13. The wrap system of claim 2, wherein the first and second wraps are further adapted to supply compression forces on the regions of the torso the wraps engage.

14. The wrap system of claim 1, wherein the system is a unitary device.

15. An adjustable heat wrap for a torso, comprising:
a. a heating wrap for wrapping around the torso of the wearer; and
b. a pair of adjustable length shoulder straps, each strap having ends connected to the upper edge of opposite ends of the wrap;
such that by adjusting the length of the shoulder straps, the heating wrap may be adjustably wrapped around a desired region of the wearer's torso.

16. A method for heat treating zones of a subject's torso, the method comprising:
a. donning onto the torso an integrated, multizone, wrap system having a lower torso wrap and a second torso wrap spaced apart from and above the lower torso wrap, both wraps having therein one or more heating elements;
b. adjusting the location of the second torso wrap to a desired zone on the torso;
c. securing the lower torso wrap and second torso wrap onto the torso; and
d. electronically activating the wrap system to supply heat to the torso.

17. The method of claim 16, wherein the activation of the wrap system supplies heat simultaneously from the lower torso wrap and second torso wrap to the torso.

18. The method of claim 16, wherein the activation of the wrap system supplies heat selectively from the lower torso wrap and second torso wrap to the torso.
